(19) United States
(12) Patent Application Publication
(10) Pub. No.: US 2018/0305329 A1
(43) Pub. Date: Oct. 25, 2018

(54) XANTHINE COMPOUND AND USE THEREOF

(71) Applicant: SUMITOMO CHEMICAL COMPANY, LIMITED, Chuo-ku (JP)

(72) Inventors: Koichiro HARADA, Osaka-shi, Osaka (JP); Tomoyuki TAKAKU, Osaka-shi, Osaka (JP); Hayato TAKEUCHI, Osaka-shi, Osaka (JP)

(21) Appl. No.: 15/770,169

(22) PCT Filed: Oct. 21, 2015
(86) PCT No.: PCT/JP2015/005310

§ 371 Date: Apr. 20, 2018

(51) Int. Cl. C07D 311/82; C09B 11/28; C12Q 1/26

(52) U.S. Cl. CPC C07D 311/82; C09B 11/28; G01N 2333/90245; G01N 2333/80; C12Q 1/26

(57) ABSTRACT

A fluorescent probe for measurement of CYP3A activity, having an excellent CYP molecular species selectivity and detection sensitivity represented is represented by the following formula:

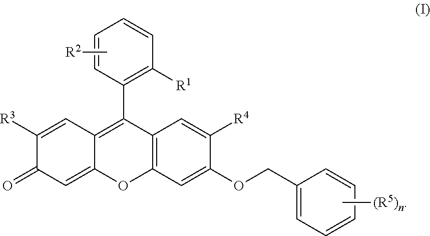

In the formula, R¹ represents a monovalent group, R² represents a hydrogen atom or a monovalent group, R³ and R⁴ each independently represent a hydrogen atom, a halogen atom, an alkyl group or an alkoxy group, R⁵ represents a monovalent group selected so that the ether bond of the O-benzyl moiety at the 6th position of the compound represented by formula (I) is oxidatively cleavable by the molecular species 3A of the cytochrome P-450, n represents an integer from 1 to 5, and when n is 2 or more, all or a part of the plurality of R⁵s may be the same as each other or different from each other.

### TECHNICAL FIELD

The present invention relates to a xanthene compound and applications thereof.

### BACKGROUND ART

Cytochrome P-450 (hereinafter, may be referred to as CYP) is an enzyme that plays a central role in the oxidative metabolism of not only endogenous substances such as steroids and fatty acids but also chemical substances such as medicines, agricultural chemicals, food additives or environmental pollutants. CYP is also involved in the development of the activities or toxicities and carcinogenicities of these chemical substances. CYP is widely distributed in the tissues of many mammals, whose expression level sometimes varies by administration of chemical substances. It has been revealed that there are many molecular species of CYP whose structures, substrate specificities, or susceptibilities to inducers differ from each other. There is a need to grasp the involvement of the CYP molecular species in evaluations of the activities and toxicities of various chemical substances. Thus, the development of a method that can measure CYP molecular species specific activities has been demanded.

The molecular species 3A of cytochrome P-450 (hereinafter, may be referred to as CYP3A) is a major molecular species involved in the metabolism of a wide variety of chemical substances.

The activity of CYP3A has been measured by a method that determines quantity of a product of CYP3A-mediated testosterone 6β-hydroxylation or a CYP3A-mediated midazolam 1-hydroxylation, using LC-MS/MS. However, these methods require a number of steps and time in preparation of a sample before a MS measurement and in the MS measurement.

An enzyme activity measuring method utilizes a fluorescent probe which is metabolized specifically by the CYP molecular species. In this method, the fluorescent probe is metabolized by CYP generate a fluorescent molecule, and a fluorescence intensity of the fluorescent molecule is measured. With this method, a large number of samples can be measured simultaneously on a multi-well plate. As the fluorescent probe for measurement of the CYP3A activity, 7-benzyloxy-4-trifluoromethylcoumarin (BFC) is commercially available, which is known to be metabolized as the substrates of CYP1A and CYP2B in addition to CYP3A. Non Patent Literature 1 describes 2,5-bis(trifluoro-methyl)-7-benzyloxy-4-trifluoromethylcoumarin (BFBFC), an analog of BFC, as a fluorescent probe specific to CYP3A.

Xanthene-based fluorescent molecules have better fluorescence quantum yields in intensity than coumarin-based fluorescent molecules. Patent Literature 1 describes a compound in which a β-galactopyranosyl group is introduced to the hydroxy group at the 6th position of the xanthene ring moiety in a xanthene-based fluorescent molecule, which can be used as a fluorescent probe for the measurement of the activity of β-galactosidase.

### CITATION LIST

### Patent Literature

[Patent Literature 1] WO2005/024049 A1

### Non Patent Literature

[Non Patent Literature 1] Xenobiotica, 2001, Vol. 31, No. 12, 861.

### SUMMARY OF INVENTION

### Technical Problem

It has been desired to develop a fluorescent probe for measurement of CYP3A activity, which is highly selective to CYP molecular species as a substrate specific to CYP3A and has superior detection sensitivity.

### Solution to Problem

The present invention provides a compound in which the hydrogen atom of the hydroxyl group at the 6th position of the xanthene ring moiety in 6-hydroxy-9-aryl-xanthenone, a xanthene-based fluorescent molecule, is replaced by benzyl group optionally substituted, an application of the compound as a fluorescent probe for the measurement of the CYP3A activity, and the like.

Specifically, the present invention provides following inventions and the like:

[1] A compound represented by the following formula (I) or a solvate thereof:

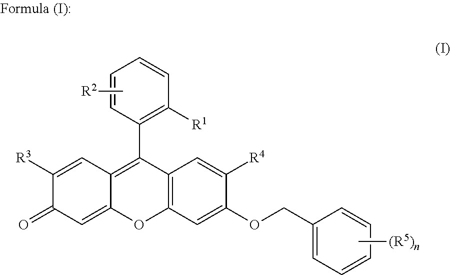

wherein R¹ represents a monovalent group; R² represents a hydrogen atom or a monovalent group; R³ and R⁴ each independently represent a hydrogen atom, a halogen atom, an alkyl group, or an alkoxy group; R⁵ represents a monovalent group which is selected so that the ether bond of the O-benzyl moiety at the 6th position of the compound represented by formula (I) is oxidatively cleavable by the molecular species 3A of cytochrome P-450; n represents an integer from 1 to 5, and when n is 2 or more, all or a part of the plurality of R⁵s may be the same or different from each other.
[2] The compound of [1], wherein R¹ and R² are independently an alkyl group, an alkenyl group, an alkynyl group, an alkoxy group, a nitro group, an amino group, a cyano group, an alkoxycarbonyl group, an alkanoylamino group, an aryl group, a heteroaryl group, an aroylamino group, or a heteroaroylamino group, wherein any hydrogen atom included in the alkyl group, the alkenyl group, the alkynyl group, the alkoxy group, the alkoxycarbonyl group, the alkanoylamino group, the aryl group, the heteroaryl group, the aroylamino group, or the heteroaroylamino group may be optionally substituted.
[3] The compound of [1] or [2], wherein R¹ is an alkyl group having 1 to 6 carbon atoms, wherein any hydrogen atom of the alkyl group may be replaced by a halogen atom; R² is an alkoxy group having 1 to 6 carbon atoms, wherein any hydrogen atom of the alkoxy group may be replaced by a halogen atom, carboxyl groups, or alkoxycarbonyl groups.
[4] The compound of any one of [1] to [3], wherein R⁵ is a hydrogen atom, a halogen atom, an alkyl group, a haloalkyl group, a nitro group, or a cyano group.
[5] The compound of any one of [1] to [4], wherein R⁵ is an alkyl group having 1 to 6 carbon atoms or a haloalkyl group having 1 to 6 carbon atoms.
[6] The compound of any one of [1] to [5], wherein n is 1 or 2.
[7] The compound of any one of [1] to [6], wherein R³ and R⁴ are hydrogen atoms.
[8] 9-(4-methoxy-2-methylphenyl)-6-bis(2,5-trifluoromethyl)benzyloxy-3H-xanthen-3-one.
[9] A use of the compound of any one of [1] to [8] as a fluorescent probe for the measurement of the CYP3A activity.
[10] A method for measuring an activity of a molecular species 3A of cytochrome P-450, comprising:
(1) reacting the compound of any one of [1] to [8] with the molecular species 3A of cytochrome P-450, and
(2) measuring the fluorescence intensity of the reaction product obtained from the step (1).
[11] The method of [10], wherein the measurement of the fluorescence intensity of the reaction product in the step (2) is performed by measuring the fluorescence intensity of the reaction solution after the reaction of the step (1), and which further comprises comparing the intensity measured in the step (2) with the fluorescence intensity measured for a control reaction solution.
[12] The method of [11], wherein the control reaction solution is the reaction solution of the step (1) before the reaction, or a reaction solution which consists of the same components as the reaction solution of the step (1) except for not containing the compound of any one of [1] to [8] or not containing the molecular species 3A of cytochrome P-450.

### Advantageous Effects of Invention

The present invention can provide a fluorescent probe for the measurement of CYP3A activity, which is highly specific to CYP molecular species and has superior detection sensitivity, a method for measuring CYP3A activity using the fluorescent probe, and the like.

### DESCRIPTION OF EMBODIMENTS

Hereinafter, the present invention is described in detail.

The term “alkyl” as used herein means a linear or branched saturated hydrocarbon having a specific number of carbon atoms, in which one or more hydrogen atoms may be independently replaced by a halogen atom or a substituent included in the present invention. Examples of the “alkyl” include alkyl having 1 to 6 carbon atoms, and specifically include methyl, ethyl, propyl, isopropyl, butyl, isobutyl, n-butyl, t-butyl, pentyl, isopentyl, n-pentyl, and hexyl.

The term “alkenyl” means a linear or branched aliphatic hydrocarbon having 2 or more carbon atoms and one or more carbon-carbon double bonds, in which one or more hydrogen atoms may be independently replaced by a halogen atom or a substituent included in the present invention. Examples of “alkenyl” include alkenyl having 2 to 6 carbon atoms, and specifically include ethenyl, propenyl, butenyl, pentenyl, and hexenyl.

The term “alkynyl” means a linear or branched aliphatic hydrocarbon having 2 or more carbon atoms and one or more carbon-carbon triple bonds, in which one or more hydrogen atoms may be independently replaced by a halogen atom or a substituent included in the present invention. Examples of the “alkynyl” include alkynyl having 2 to 6 carbon atoms, and specifically include ethynyl, propynyl, butynyl, pentynyl, and hexynyl.

Herein, “alkoxy” means a group represented by —ORa, wherein “Ra” represents an alkyl defined above. Examples of the “alkoxy” include methoxy.

Herein, “halogen” means fluorine, chlorine, bromine or iodine.

The term “aryl” means an aromatic group composed of carbon atoms and hydrogen atoms, or a monocyclic or polycyclic aromatic group containing at least one hetero atom selected from a group consisting of a nitrogen atom, an oxygen atom and a sulfur atom, in which one or more hydrogen atoms may be independently replaced by a halogen atom or a substituent included in the present invention. Examples of “aryl” include aryl having 6 to 10 carbon atoms, and specifically include a phenyl group, a biphenyl group, a naphthyl group, a thienyl group, a furyl group, a pyrrolyl group, a pyrazolyl group, an isothiazolyl group, an isoxazolyl group, a pyridyl group, a pyrazinyl group, a pyrimidinyl group, and a pyridazinyl group. The same shall apply to the aryl or heteroaryl moiety of the groups having the aryl or a heteroaryl moiety (such as an aroyl group and a heteroaroyl group).

The term “haloalkyl” as used herein means an alkyl group defined herein in which at least one hydrogen atom is replaced by a halogen atom. Specific examples of the branched or linear “haloalkyl” useful in the present invention include methyl, ethyl, propyl, isopropyl, n-butyl and t-butyl in which one or more hydrogen atoms are independently replaced by a halogen atom (such as a fluorine atom, a chlorine atom, a bromine atom or an iodine atom). More specific examples thereof include —CF3, and —CH2—CH2—F.

The phrase “optionally substituted” means that at least one of the hydrogen atoms contained in an object group may be replaced any of the above-described halogen atoms or other substituents.

The term “solvate” as used herein means various stoichiometric complexes between the solute (in the present invention, the compound represented by formula (I)) and the solvent. According to the object of the present invention, the solvent should not disturb the useful functions of the solute. Specific examples of the appropriate solvent include water, methanol and ethanol.

In formula (I), R¹ represents a monovalent group.

Examples of the monovalent group include an alkyl group, an alkenyl group, an alkynyl group, an alkoxy group, a nitro group, an amino group, a cyano group, an alkoxycarbonyl group, an alkanoylamino group, an aryl group, a heteroaryl group, an aroylamino group, and a heteroaroylamino group, wherein hydrogen atom(s) contained in said alkyl group, alkenyl group, alkynyl group, alkoxy group, alkoxycarbonyl group, alkanoylamino group, aryl group, heteroaryl group, aroylamino group and heteroaroylamino group may be replaced.

As R¹, an alkyl group having 1 to 6 carbon atoms is preferable, wherein one or more hydrogen atoms may be independently replaced by a halogen atom.

In formula (I), R² represents a hydrogen atom or a monovalent group.

Examples of the monovalent group include an alkyl group, an alkenyl group, an alkynyl group, an alkoxy group, a nitro group, an amino group, a cyano group, an alkoxycarbonyl group, an alkanoylamino group, an aryl group, a heteroaryl group, an aroylamino group, and a heteroaroylamino group, wherein hydrogen atom(s) contained in said alkyl group, alkenyl group, alkynyl group, alkoxy group, alkoxycarbonyl group, alkanoylamino group, aryl group, heteroaryl group, aroylamino group and heteroaroylamino group may be replaced.

As R², an alkoxy group having 1 to 6 carbon atoms is preferable, wherein one or more hydrogen atoms may be replaced by a halogen atom, a carboxyl group or an alkoxycarbonyl group. Examples of the substituted alkoxy group of R² include a carboxyl-substituted C₁₋₆ alkoxy group and an alkoxycarbonyl-substituted C₁₋₆ alkoxy group.

In formula (I), R³ and R⁴ independently represent a hydrogen atom, a halogen atom, an alkyl group or an alkoxy group.

As R³ and R⁴, a hydrogen atom is preferable.

In formula (I), each of R⁵ independently represents a monovalent group which is selected so that the ether bond of the O-benzyl moiety at the 6th position of the compound represented by formula (I) is oxidatively cleavable by the molecular species 3A of cytochrome P-450a monovalent group. When n is 2 or more, all or a part of the plurality of R⁵s may be the same or different from each other.

Examples of such a monovalent group may include a hydrogen atom, a halogen atom, an alkyl group, a haloalkyl group, a nitro group and a cyano group.

As R⁵, an alkyl group having 1 to 6 carbon atoms or a haloalkyl group having 1 to 6 carbon atoms is preferable.

Examples of the compound represented by formula (I) may include:

a compound in which R¹ is an alkyl group having 1 to 6 carbon atoms, R² is an alkoxy group having 1 to 6 carbon atoms, R³ and R⁴ are hydrogen atoms, R⁵ is a haloalkyl group having 1 to 6 carbon atoms, and n is 2; and

a compound in which R¹ is a methyl group, R² is a methoxy group, R³ and R⁴ are hydrogen atoms, R⁵ is a trifluoromethyl group, and n is 2.

More specific examples of the compound represented by formula (I) may include 9-(4-methoxy-2-methylphenyl)-6-bis(2,5-trifluoromethyl)benzyloxy-3H-xanthen-3-one.

Specific compounds described herein contain one or more chiral centers and can exist as a plurality of stereoisomers. Mixtures of stereoisomers and purified enantiomers or enantiomerically/diastereomerically enriched mixtures are also included in the present invention. The individual isomers of the compound of formula (I), and the completely or partially equilibrated mixtures of the isomers are also included in the present invention. The individual isomers of the compound of formula (I) in the mixtures with the corresponding isomer whose one or more chiral centers are inverted are also included in the present invention. Specific enantiomers can be separated and collected by the techniques known in the art such as chromatography in chiral stationary phase or chiral salt formation followed by separation based on selective crystallization. By using a specific enantiomer as a starting substance, it is also possible to obtain a corresponding isomer as the final product.

The compound of formula (I) may be in a form of a crystal. Both of a single crystal form and a crystal form mixture are also included in the compounds of formula (I). The crystal can be produced by crystallizing the compound by applying known crystallization methods. The compounds labeled with isotopic elements (such as ³H, ¹⁴C, ¹⁸F, ³⁵S, and ¹²⁵I) or the like are also included in the compounds of formula (I).

The compound of formula (I) can be crystallized in two or more forms known as polymorphism, and such polymorphic forms (polymorph) are included in the compound of formula (I). In general, the polymorphism can be generated in response to the variation of the temperature or pressure, or both of them. The polymorphism can also be generated by the fluctuation in the crystallization process. The polymorphism can be discriminated by the various physical features known in the art such as X-ray diffraction pattern, solubility and melting point.

Hereinafter, a method for producing the compound represented by formula (I) (hereinafter, may be referred to as the compound (I)) or a solvate thereof.

The compound (I) or a solvate thereof can be produced, for example, by the reaction of following scheme. The reaction is, for example, the Williamson reaction, one of the common synthetic reactions for synthesizing ethers. The compound (I) can be synthesized by reacting the phenolic hydroxy group of the xanthene-based fluorescent molecule (A) with a benzyl halide (X represents a halogen atom) in the presence of a base. As the halogen (X), bromine or chlorine is preferable. Examples of the base include potassium carbonate, cesium carbonate, sodium hydroxide, sodium hydride, silver(I) carbonate, and silver(I) oxide. The reaction solvent is preferably aprotic polar solvents such as DMF, DMSO, and acetonitrile.

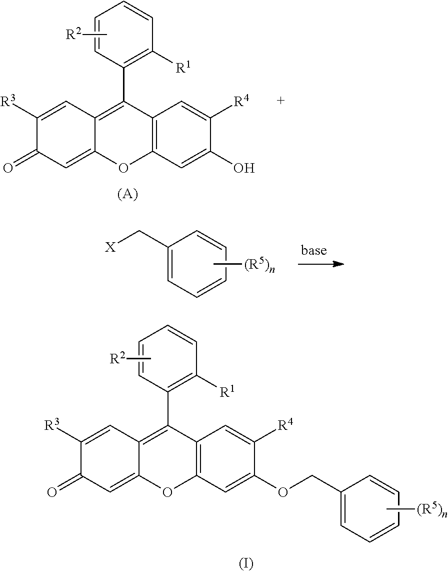

The compound represented by formula (I) or a solvate thereof (hereinafter, may be collectively referred to as the compound of the present invention) can be used as a fluorescent probe for measurement of the CYP3A activity. The compound of the present invention is a substrate specific to CYP3A, and produces an intensely fluorescent xanthene-based fluorescent molecule by oxidative metabolism in the coexistence of the enzyme. For example, the compound of the present invention (9-(4-methoxy-2-methylphenyl)-6-bis(2,5-trifluoromethyl)benzyloxy-3H-xanthen-3-one) in which the hydrogen atom of the hydroxy group of 6-hydroxy-9-(4-methoxy-2-methylphenyl)-3H-xanthen-3-one is replaced by 2,5-bis(trifluoromethyl)benzyl group emits only a weak fluorescence in response to the irradiation of an excitation light at a wavelength of 482 nm. On the other hand, in the products of the reaction between the compound of the present invention and CYP3A, an extremely intense fluorescence (wavelength: 520 nm) can be observed due to 6-hydroxy-9-(4-methoxy-2-methylphenyl)-3H-xanthen-3-one which is generated by metabolism. Accordingly, the CYP3A enzyme activity can be quantitatively determined by reacting the compound of the present invention as a substrate with CYP3A, and measuring the fluorescence of the intensely fluorescent molecule produced by metabolism of the compound of the present invention. By using the compound of the present invention as a fluorescent probe, the CYP3A enzyme activities of a large number of samples can be simultaneously measured on a multiwell plate.

The method for measuring CYP3A activity of the present invention comprises:

(1) reacting the compound of the present invention with CYP3A; and

(2) measuring the fluorescence intensity of the reaction product obtained from the step (1).

The compound of the present invention is reacted as a substrate with CYP3A, the fluorescence intensity emitted from the fluorescent molecule generated by oxidative metabolization of the compound of the present invention is measured to quantify the fluorescent molecule, and thus the CYP3A activity is measured. If necessary, the fluorescence intensities of the reaction solutions measured before and after the reaction of the compound of the present invention with CYP3A may be compared, or the fluorescence intensities measured after the reaction with CYP3A may be compared between the compound of the present invention and a control substance. For example, in the step (2), the fluorescence intensity of the reaction product is measured for the reaction solution after the reaction of the step (1), and the value measured in the step (2) is compared with the fluorescence intensity of the control reaction solution. Examples of such a control reaction solution may include the reaction solution before the reaction of the step (1), or a reaction solution consisting of the same composition as the reaction solution used in the step (1) except that the compound of the present invention or CYP3A is not contained.

The reaction of the compound of the present invention with CYP3A can be performed in a reaction solution composition and under a reaction condition which are usually employed for the measurement of the metabolic rate of a compound by CYP, under the coexistence of NADPH-P450 reductase and NADPH, a coenzyme thereof. The concentration of the compound of the present invention in the reaction solution may be about 0.1 μM to about 100 μM. The amount of CYP3A added to the reaction solution may be about 0.1 pmol to about 100 pmol. The reaction temperature is usually within the range from about 20° C. to about 37° C.

The measurement of the CYP3A enzyme activity by the method of the present invention can be performed under a neutral condition, for example, within the range from about pH 5 to about pH 9, preferably within a range from about pH 6 to about pH 8, and more preferably within a range from about pH 6.8 to about pH 7.6.

As the fluorescent probe for measuring the CYP3A activity, the compound of the present invention may be used as it is, or if necessary, may be used as a composition containing additives usually used in the preparation of a reagent. For example, additives for the reagent to be used in a physiological environment such as a solubilizing agent, a pH regulator, a buffering agent, a tonicity agent or the like may be blended with the compound of the present invention. The amounts of these additives blended can be appropriately selected by those skilled in the art. These compositions can be provided as compositions in appropriate forms such as a mixture in powder form, a lyophilizate, a granule, a tablet, and a liquid agent.

### EXAMPLES

Hereinafter, the present invention is described in more detail by way of Examples and Test Example, but the present invention is not limited thereto.

In Examples, the following abbreviations may be used:

NMR (nuclear magnetic resonance); g (gram); mg (milligram); mL (milliliter); μL (microliter); mmol (millimol); pmol (picomol); mM (millimolar concentration); μM (micromolar concentration); N (normal concentration); nm (nanometer); Hz (Hertz); eq (molar equivalent); THF (tetrahydrofuran); DMF (N,N-dimethylformamide); DMSD (dimethyl sulfoxide); CDCl₃ (deuterated chloroform); DMSD-d₆ (deuterated dimethyl sulfoxide); Mg (magnesium); NaHCO₃ (sodium hydrogen carbonate); MgSO₄ (magnesium sulfate); TBDMSCl (t-butyldimethylsilyl chloride); TBDMSO {[t-butyl(dimethyl)silyl]oxy}; NADPH (nicotinamide adeninedinucleotide phosphate/reduced form).

Various data were measured by using the following analytical instruments.

¹H-NMR: AV300M, AV600 (BRUKER)

Quantum Efficiency (QE): QE-1100 (Otsuka Electronics Co., Ltd.)

Microplate reader: Safire2 (TECAN)

For the ¹H-NMR spectra, the chemical shifts are shown in ppm (ppm, δ values) units, and the coupling constants are shown in Hertz (Hz, J values) units. The splitting parameters show the apparent multiplicities, which are denoted as s (singlet), d (doublet), t (triplet), q (quartet), m (multiplet) or br (broad).

### Example 1-1: Production of 3,6-dihydroxy-9H-xanthen-9-one

In a reactor (Storage bottle, ACE GLASS), 6.00 g (24.4 mmol) of 2,2′,4,4′-tetrahydroxybenzophenone and 40 mL of distilled water were placed, which was heated at between 195° C. and 200° C. for 4 hours. The reaction mixture was cooled to room temperature, and then the precipitated crude product was collected by filtration, which was subsequently added to 50 mL of distilled water and recrystallized under reflux conditions to obtain 5.18 g (yield: 93%) of 3,6-dihydroxy-9H-xanthen-9- as a pale yellow crystal.

¹H-NMR (300 MHz, DMSO-d₆): δ 6.82-6.89 (m, 4H), 8.26 (d, J=8.7 Hz, 2H), 10.83 (brs, 2H).

### Example 1-2: Production of 3,6-bis{[t-butyl(dimethyl)silyl]oxy}-9H-xanthen-9-one

In 25 mL of DMF, 4.95 g (21.7 mmol) of 3,6-dihydroxy-9H-xanthen-9-one obtained according to Example 1-1 and 7.39 g (5.0 eq) of imidazole were dissolved, to which 8.18 g (2.5 eq) of t-butyldimethylsilyl chloride was added under cooling with ice, which was stirred in a nitrogen atmosphere at room temperature for 5 hours. The reaction mixture was poured into water and extracted with ethyl acetate. The obtained organic layer was washed with saturated saline and then dried with MgSO₄. The solvent was evaporated under reduced pressure, and the residue was purified by silica gel column chromatography (eluent: hexane/ethyl acetate=3/1) to obtain 9.07 g (yield: 95%) of 3,6-bis{[t-butyl(dimethyl)silyl]oxy}-9H-xanthen-9-one as a white crystal.

¹H-NMR (300 MHz, CDCl₃): δ 0.34 (s, 12H), 1.08 (s, 18H), 6.74-6.79 (m, 4H), 8.10 (dd, J=1.5, 7.5 Hz, 2H).

### Example 1-3: Production of 6-hydroxy-9-(4-methoxy-2-methylphenyl)-3H-xanthen-3-one (Hereinafter, May Referred to as the Compound (1))

In a reactor, 165 mg (2.0 eq) of Mg and 10 mL of THF were placed, to which 1.37 g (2.0 eq) of 2-bromo-5-methoxytoluene was added under nitrogen atmosphere, which was stirred at 60° C. for 5 hours. The reaction mixture was cooled in an ice bath, followed by addition of drop of 10 mL of a THF solution of 1.50 g (3.40 mmol) of 3,6-bis{[t-butyl(dimethyl)silyl]oxy}-9H-xanthen-9-one obtained according to Example 1-2, which was returned to room temperature and stirred for 30 minutes. Subsequently, 20 mL of a 2 N hydrochloric acid aqueous solution was gradually added to the reaction mixture under cooling with ice, which was stirred at room temperature for 15 hours, and then added chloroform. The organic layer was separated and washed sequentially with a saturated NaHCO₃ aqueous solution and a saturated saline. The washed organic layer was dried with MgSO₄, and the solvent was evaporated under reduced pressure. Obtained crystalline residue was washed with diethyl ether, which gave 0.77 g (yield: 68%) of 6-hydroxy-9-(4-methoxy-2-methylphenyl)-3H-xanthen-3-one as an orange crystal.

¹H-NMR (300 MHz, DMSO-d₆) : δ 1.98 (s, 3H), 3.86 (s, 3H), 6.96 (d, J=9.2 Hz, 2H), 7.03 (m, 3H), 7.10 (s, 1H), 7.23 (d, J=8.2 Hz, 1H), 7.28 (d, J=9.2 Hz, 2H).

The synthetic scheme of the compound (1) is shown below.

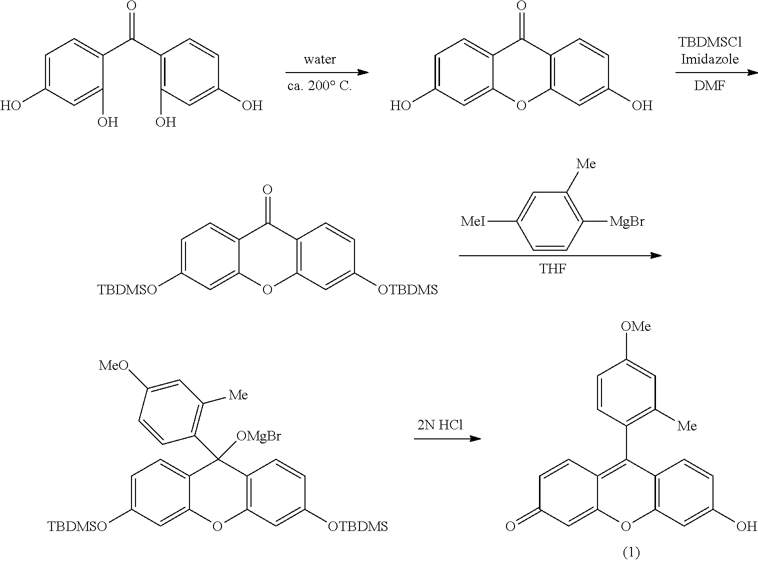

### Example 2: Production of 9-(4-methoxy-2-methylphenyl)-6-bis(2,5-trifluoromethyl)benzyloxy-3H-xanthen-3-one (Hereinafter, Sometimes Denoted as the Compound (2))

In 5 mL of DMF, 200 mg (0.60 mmol) of 6-hydroxy-9-(4-methoxy-2-methylphenyl)-3H-xanthen-3-one obtained according to Example 1-3 was dissolved, to which 26 mg (1.1 eq) of sodium hydride in oil was added under cooling with ice, and stirred at room temperature for 30 minutes. To the resulting reaction mixture, 2,5-bis(trifluoromethyl)benzyl bromide was added under cooling with ice, and stirred at the same temperature for 30 minutes. Then, the reaction temperature was returned to the room temperature, and the mixture was further stirred for 1 hour. The reaction mixture was poured into a saturated ammonium chloride aqueous solution, which was extracted with ethyl acetate, and the organic layer was washed with a saturated saline and then dried with MgSO₄. The solvent was evaporated under reduced pressure, and obtained residue was purified by silica gel column chromatography (eluent: hexane/ethyl acetate=3/1) to yield 296 mg (yield: 88%) of 9-(4-methoxy-2-methylphenyl)-6-bis(2,5-trifluoromethyl)benzyloxy-3H-xanthen-3-one was obtained as an orange crystal.

¹H-NMR (400 MHz, CDCl₃): δ 2.06 (s, 3H), 3.90 (s, 3H), 5.39 (s, 2H), 6.45 (d, J=2.0 Hz, 1H), 6.58 (dd, J=2.0, 10.0 Hz, 1H), 6.86-6.99 (m, 3H), 7.01-7.04 (m, 2H), 7.07-7.10 (m, 2H), 7.76 (d, J=8.0 Hz, 1H), 7.89 (d, J=8.0 Hz, 1H), 8.02 (s, 1H).

The synthetic scheme of the compound (2) is shown below.

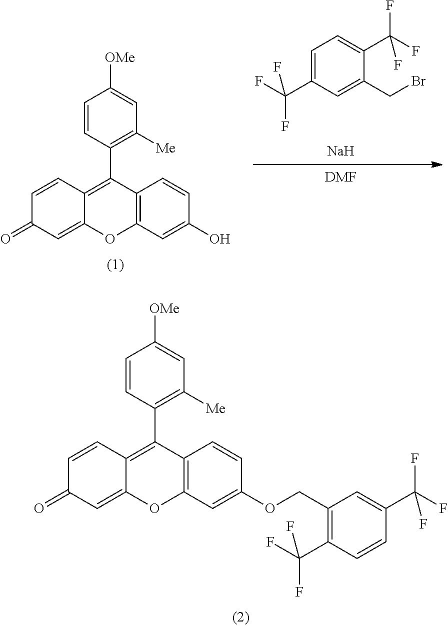

The spectroscopic properties of the compound (1), the compound (2) and trifluoromethyl-7-hydroxycoumarin (FHC) are shown in Table 1.

TABLE 1

| | Compound | | |
|---|---|---|---|
| | Compound (1) | Compound (2) | FHC |
| Excitation wavelength | 492 nm | 460 nm | 405 nm |
| Emission wavelength | 509 nm | 519 nm | 498 nm |
| Quantum Efficiency | 85.3% | 20.6% | 53.0% |

### Test Example 1: Metabolism Test (Molecular Species Selectivity Evaluation) of the Compound of the Present Invention by Using Human and Rat CYP Molecular Species

A 100 mM phosphate buffer solution (pH 7.4) containing 3 μM of the compound (2) synthesized according to Example 2, 1 mM of NADPH (Wako Pure Chemical Industries, Ltd.) and 0.1% of DMSO was prepared. In each of the wells of a 96-well plate, 2 μL of each microsome from recombinant insect cell expressing different type of molecular species of CYP (CYP 2 pmol, BD Supersomes™, Nippon BD) was placed, followed by addition of 198 μL of the above-described phosphate buffer solution to start the reaction, which was incubated at 37° C. for 20 minutes. The reaction was stopped by adding 100 μL of acetonitrile (Nacalai Tesque, Inc.), and the fluorescence intensity of the obtained reaction solution was measured at the fluorescence wavelength of 520 nm with an excitation wavelength of 482 nm. A calibration curve was prepared by using the 0 μM, 0.01 μM, 0.05 μM, 0.1 μM, 0.50 μM and 1 μM solutions (100 mM phosphate buffer/acetonitrile=2/1) of the compound (1). The amounts of the compound (1) produced by reacting the compound (2) with the different types of molecular species of human CYP are shown in Table 2, and the amounts of the compound (1) produced by reacting the compound (2) with the different types of molecular species of rat CYP are shown in Table 3, It has been revealed that the compound (2) is a substrate specific to human and rat CYP3A, and the CYP3A enzyme activity can be measured by measuring the fluorescence intensity of the product obtained by the reaction of the compound (2) with CYP3A.

TABLE 2

| Human CYP isoform | Compound (1) (pmol/min/mg protein)* |
|---|---|
| CYP1A2 | N.D. (3) |
| CYP2A6 | N.D. (3) |
| CYP2B6 | N.D. (3) |
| CYP2C19 | N.D. (3) |
| CYP2C8 | N.D. (3) |
| CYP2C9 | N.D. (3) |
| CYP2D6 | N.D. (3) |
| CYP2E1 | 0.0038 ± 0.0052 (3) |
| CYP3A4 | 2.4913 ± 0.0497 (3) |

*Mean ± SD is shown, and N.D. means “not detected”. The numbers in parentheses indicate the repetition numbers.

TABLE 3

| Rat CYP isoform | Compound (1) (pmol/min/mg protein)* |
|---|---|
| CYP1A2 | N.D. (3) |
| CYP2A1 | N.D. (3) |
| CYP2A2 | N.D. (3) |
| CYP2B1 | N.D. (3) |
| CYP2C6 | N.D. (3) |
| CYP2C11 | N.D. (3) |
| CYP2C12 | N.D. (3) |
| CYP2C13 | N.D. (3) |
| CYP2D1 | 0.0151 ± 0.0081 (3) |
| CYP2D2 | N.D. (3) |
| CYP2E1 | N.D. (3) |
| CYP3A1 | 0.2190 ± 0.0095 (3) |
| CYP3A2 | 0.0912 ± 0.0105 (3) |

*Mean ± SD is shown, and N.D. means “not detected”. The numbers in parentheses indicate the repetition numbers.

### INDUSTRIAL APPLICABILITY

The present invention can provide a fluorescent probe for the measurement of the CYP3A activity, having an excellent CYP molecular species selectivity and detection sensitivity, a method for measuring the CYP3A activity using the fluorescent probe, and the like.

1. A compound represented by the following formula (I) or a solvate thereof: 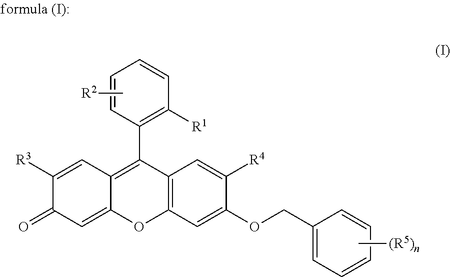
wherein R¹ represents a monovalent group; R² represents a hydrogen atom or a monovalent group; R³ and R⁴ each independently represent a hydrogen atom, a halogen atom, an alkyl group, or an alkoxy group; R⁵ represents a monovalent group selected so that the ether bond of the O-benzyl moiety at the 6th position of the compound represented by formula (I) is oxidatively cleavable by the molecular species 3A of the cytochrome P-450, n represents an integer from 1 to 5, and when n is 2 or more, all or a part of the plurality of R⁵s may be the same or different from each other.

2. The compound of claim 1, wherein R¹ and R² are independently an alkyl group, an alkenyl group, an alkynyl group, an alkoxy group, a nitro group, an amino group, a cyano group, an alkoxycarbonyl group, an alkanoylamino group, an aryl group, a heteroaryl group, an aroylamino group, or a heteroaroylamino group, wherein any hydrogen atom included in the alkyl group, the alkenyl group, the alkynyl group, the alkoxy group, the alkoxycarbonyl group, the alkanoylamino group, the aryl group, the heteroaryl group, the aroylamino group, or the heteroaroylamino group may be optionally substituted.

3. The compound of claim 1 or 2, wherein R¹ is an alkyl group having 1 to 6 carbon atoms, wherein any hydrogen atom of the alkyl group may be replaced by a halogen atom; R² is an alkoxy group having 1 to 6 carbon atoms, wherein any hydrogen atom Of the alkoxy group may be replaced by a halogen atom, a carboxyl group, or an alkoxycarbonyl group.

4. The compound of any one of claims 1 to 3, wherein R⁵ is a hydrogen atom, a halogen atom, an alkyl group, a haloalkyl group, a nitro group, or a cyano group.

5. The compound of any one of claims 1 to 4, wherein R⁵ is an alkyl group having 1 to 6 carbon atoms or a haloalkyl group having 1 to 6 carbon atoms.

6. The compound of any one of claims 1 to 5, wherein n is 1 or 2.

7. The compound of any one of claims 1 to 6, wherein R³ and R⁴ are hydrogen atoms.

8. 9-(4-Methoxy-2-methylphenyl)-6-bis(2,5-trifluoromethyl)benzyloxy-3H-xanthen-3-one.

9. A use of the compound of any one of claims 1 to 8 as a fluorescent probe for the measurement of the CYP3A activity.

10. A method for measuring the activity of the molecular species 3A of cytochrome P-450, comprising:
(1) reacting the compound of any one of claims 1 to 8 with the molecular species 3A of cytochrome P-450, and
(2) measuring the fluorescence intensity of the reaction product obtained from the step (1).

11. The method of claim 10, wherein the measurement of the fluorescence intensity of the reaction product in the step (2) is performed by measuring the fluorescence intensity of the reaction solution after the reaction of the step (1), and which further comprising comparing the intensity measured in the step (2) with the fluorescence intensity for a control reaction solution.

12. The method of claim 11, wherein the control reaction solution is the reaction solution of the step (1) before the reaction, or a reaction solution which consists of the same components as the reaction solution of the step (1) except for not containing the compound of any one of claims 1 to 8 or not containing the molecular species 3A of cytochrome P-450.
